(19) United States
(12) Patent Application Publication
(10) Pub. No.: US 2023/0041841 A1
(43) Pub. Date: Feb. 9, 2023

(54) SYSTEM AND METHOD FOR ADMINISTERING A MEDICAL FLUID TO A PATIENT

(71) Applicant: Fresenius Vial SAS, Brézins (FR)

(72) Inventors: Damien Barbeyrac, Veurey Voroize (FR); Rémy Wolff, Tullins (FR); Olivier Artarit, St Etienne de Crossey (FR)

(21) Appl. No.: 17/789,442

(22) PCT Filed: Feb. 19, 2021
(86) PCT No.: PCT/EP2021/054091

§ 371 Date: Jun. 27, 2022

(30) Foreign Application Priority Data
Feb. 20, 2020 (EP) 20290020.5

(51) Int. Cl. A61M 5/14; A61M 5/142

(52) U.S. Cl. CPC A61M 5/1413; A61M 5/142

(57) ABSTRACT

A system for administering a medical fluid to a patient (P), comprises an infusion station (1) comprising a rack (10) and a control device (11), the rack (10) defining a multiplicity of slots (100), wherein the control device (11) is configured to synchronize reference patient data between infusion devices (2) attached to said multiplicity of slots (100), said reference patient data relating to a patient (P) to be administered with a medical fluid using the infusion station (1), and a new infusion device (2A) to be attached to one of said multiplicity of slots (100) of the rack (10). The control device (11) herein is configured, on the occasion of attaching the new infusion device (2A) to one of the multiplicity of slots (100) of the rack (10), to receive, from the new infusion device (2A), device patient data defined on the new infusion device (2A), and to update said reference patient data based on the device patient data dependent on an infusion status of the new infusion device (2A) and on an infusion status of the infusion station (1).

### DESCRIPTION

The invention relates to a system for administering a medical fluid to a patient according to the preamble of claim 1 and to a method for administering a medical fluid to a patient.

A system of this kind comprises an infusion station comprising a rack and a control device, the rack defining a multiplicity of slots for receiving a multiplicity of infusion devices. The control device is configured to synchronize reference patient data between infusion devices attached to the multiplicity of slots, the reference patient data relating to a patient to be administered with a medical fluid using the infusion station.

A system of this kind may generally be used at the bedside of a patient in a healthcare environment, for example within an intensive care unit (ICU) of a hospital. Using an infusion station of the type described herein a multiplicity of infusion devices, such as syringe infusion pumps or volumetric (peristaltic) infusion pumps, may be combined in order to administer one or multiple medical fluids, such as medications for the treatment of the patient or nutritional solutions for the enteral or parenteral feeding of the patient, to a patient in a concerted fashion, for example within an infusion protocol in which infusion operations are carried out sequentially or in parallel. Furthermore, using the infusion station infusion devices may be organized and physically held to form for example a stack of infusion devices at the bedside of the patient, such that an organized and easy-to-handle arrangement of infusion devices is obtained. The rack herein serves as a communication link in that it links infusion devices to each other and to the control device, the rack hence serving as a communication spine which in addition may provide for a power supply for electrically feeding the infusion devices connected thereto.

Generally, a particular infusion station is used to perform infusion operations on a single patient. It herein is conceivable that multiple racks are present within an infusion station and are associated with a single control device for controlling the racks and the infusion devices arranged thereon.

An infusion operation carried out by an infusion device of the infusion station herein may depend on certain patient data relating to the patient, such as the patient’s weight, age, gender, body surface area (BSA), body mass index (BMI) and the like. Dependent on the patient data bounds and limits may be defined for an infusion operation, for example limits for a dosage and a flow rate. In addition, the determination of a dose rate may have to take patient data, such as a patient’s weight, into account.

For an infusion station there hence is a desire that patient data is synchronized between the different infusion devices connected to the rack. In particular, all infusion devices shall use the same reference patient data for carrying out an infusion operation, such that there is no mismatch between the patient data and correspondingly infusion settings applied by the different infusion devices.

The synchronization of the patient data generally takes place under the control of the control device.

US2015370973 relates to a method for transferring operational data to a medical device for administering a drug to a patient. The document refers to a group of infusion pumps installed on a rack. An infusion device can be plugged to the rack, upon which the rack validates the configuration of the attached infusion device against its reference copy. If an update of the data installed on the infusion device is necessary, the infusion device may enter an update sequence with rack, without further details provided.

Herein, it is desirable that the synchronization is easy and comfortable for a user, possibly requiring a limited number of user interactions to achieve a distribution of synchronized patient data in between the infusion devices connected to the rack of the infusion station. This in particular shall also be the case if a new infusion device is connected to the rack, potentially requiring an update of patient data on the infusion station to ensure correct infusion settings for all infusion devices connected to the infusion station.

It is an object of the instant invention to provide a system and a method for administering a medical fluid to a patient which allow for an easy synchronization of patient data in particular on the occasion of connecting a new infusion device to the rack of an infusion station.

This object is achieved by means of the system comprising the features of claim 1.

Herein, the control device is configured, on the occasion of attaching a new infusion device to one of the multiplicity of slots of the rack, to receive, from the new infusion device, device patient data defined on the new infusion device, and to update said reference patient data based on the device patient data dependent on an infusion status of the new infusion device and on an infusion status of the infusion station.

Accordingly, on the occasion of attaching a new infusion device to one of the slots of the rack an update of the reference patient data is potentially carried out, dependent on an infusion status of the new infusion device and on an infusion status of the infusion station.

When a new infusion device is connected to the rack of the infusion station a synchronization of patient data is to be carried out in order to make sure that the infusion devices already connected to the rack and the new infusion device which now is connected to the rack use the same patient data relating to the patient currently under treatment by the infusion station. The synchronization herein does not simply take place by a transfer of data from the control device towards the new infusion device, but takes into account an infusion status of both the new infusion device and the infusion station and based on this potentially updates the reference patient data as currently set on the infusion station according to the device patient data as defined on the new infusion device.

In particular, dependent on the infusion status of the new infusion device and on the infusion status of the infusion station an updating of the reference patient data of the infusion station may take place, or - alternatively - the reference patient data as previously defined on the infusion station may be transferred to the new infusion device for the purpose of synchronization. Hence, potentially, if it is found that the device patient data should have priority, the reference patient data as currently stored in the control device of the infusion station is updated by the device patient data. Alternatively, if it is found that the reference patient data as currently stored in the control device of the infusion station should have priority, the device patient data is updated by the reference patient data as stored in the control device for synchronizing with the infusion station.

The updating and synchronization may take place automatically on the occasion of attaching the new infusion device to the respective slot of the rack. Herein, the control device, in one embodiment, may be configured to identify a connection event indicating that the new infusion device is attached to one of the slots of the rack. For this, for example a sensor, such as a switch, may be present at a slot to output a detection signal on the occasion of attaching an infusion device to the slot. The updating of the reference patient data may then take place based on the device patient data subsequent (i.e., in direct reaction to) the identification of the connection event.

In one embodiment, the new infusion device is configured to transfer said device patient data to the control device on the occasion of attaching the new infusion device to one of the multiplicity of slots of the rack. The transfer of device patient data may take place automatically without a user interaction upon the detection of the connection event, such that the updating of the reference patient data for the purpose of synchronization may take place substantially without user interaction, synchronization hence becoming easy and comfortable for a user when attaching a new infusion device to an infusion station.

In one embodiment, the control device is configured to synchronize the updated reference patient data between the infusion devices attached to said multiplicity of slots. If the reference patient data as previously stored on the infusion station is updated according to the device patient data on the occasion of attaching the new infusion device to the respective slot of the rack, the updated reference patient data is distributed among the further infusion devices connected to the rack, such that all infusion devices connected to the rack obtain and are synchronized with the same reference patient data, hence making sure that the same patient data is used by all infusion devices of the infusion station.

The updating of the reference patient data as stored and defined within the control device of the infusion station takes place dependent on the infusion status of the new infusion device and the infusion status of the infusion station. Herein, the infusion status of the new infusion device in one embodiment is indicative of whether the new infusion device is currently carrying out an infusion operation. The infusion status of the new infusion device hence indicates whether, when connecting the infusion device to the rack, the infusion device is in the progress of carrying out an infusion operation or not.

Likewise, the infusion status of the infusion station may be indicative of whether at least one of the infusion devices attached to said multiplicity of slots is currently carrying out an infusion operation. The infusion station hence is assumed to be in an infusing state if one or multiple of the infusion devices connected to the rack (not counting the new infusion device) is currently in the progress of carrying out an infusion operation.

Dependent on the infusion status of the new infusion device and on the infusion status of the infusion station it is determined whether the device patient data as stored and defined in the new infusion device or the reference patient data as previously defined in the control device should have priority. Hence, dependent on the infusion status of the new infusion device and on the infusion status of the infusion station it is determined whether the reference patient data of the infusion station is updated according to the device patient data of the new infusion device or not.

The updating of the reference patient data generally may take place by amending the reference patient data according to the device patient data, for example by replacing or amending some values and leaving others unchanged, or by merging the reference patient data as stored in the control device with the device patient data. In a preferred embodiment, the reference patient data is set to the device patient data in case it is found that the device patient data should be prioritized dependent on the infusion status of the new infusion device and on the infusion status of the infusion station. Hence, in this case the reference patient data as previously stored in the control device of the infusion station is replaced by the device patient data, such that the reference patient data is updated to assume the values of the device patient data.

By considering the infusion status of the new infusion device and the infusion status of the infusion station for updating the reference patient data it is taken into account whether the new infusion device, at the time of attaching it to the rack, is infusing or not and whether the infusion station, at the time of attaching the new infusion device to the rack, is infusing or not.

For example, the control device may be configured to set the reference patient data to the device patient data if the infusion status of the new infusion device indicates that the new infusion device is currently carrying out an infusion operation, and if in addition the infusion status of the infusion station indicates that none of the infusion devices attached to said multiplicity of slots is carrying out an infusion operation. Hence, the device patient data is assumed to have a higher priority if the new infusion device is, at the time of attaching it to the rack, infusing and the infusion station is not infusing. In this case, the reference patient data in the control device of the infusion station is set to the device patient data as received from the new infusion device, such that the reference patient data is updated according to the device patient data received from the new infusion device. Following the updating of the reference patient data, the reference patient data may be synchronized between all infusion devices connected to the rack and, for this, may be distributed to all infusion devices such that all infusion devices are set to the same reference patient data.

Furthermore, the control device may be configured to not update the reference patient data as previously stored in the control device if the infusion status of the new infusion device indicates that the new infusion device is currently not carrying out an infusion operation, or if the infusion status of the infusion station indicates that at least one of the infusion devices attached to said multiplicity of slots is currently carrying out an infusion operation. If the new infusion device is currently not infusing, it always is assumed that the reference patient data as previously stored in the control device should have priority. In addition, if the infusion station is found to be infusing, i.e. one or multiple of the infusion devices connected to the rack (not counting the new infusion device) are currently carrying out an infusion operation, it is assumed that the reference patient data as stored in the control device should have priority, independent on the infusion status of the new infusion device. Hence, if the reference patient data as previously stored in the control device is assumed to have priority over the device patient data as stored in the new infusion device, the reference patient data of the infusion station is not updated and hence is not changed, but remains at the previously stored values.

If the reference patient data is not updated, the reference patient data is transferred to the new infusion device such that the new infusion device obtains the reference patient data as previously stored in the control device of the infusion station and hence is synchronized with the infusion station.

In case the new infusion device is currently carrying out an infusion operation when it is attached to the infusion station, a conflict may arise in between the settings of the new infusion device for the current infusion operation and the newly received reference patient data, in case the reference patient data as previously stored in the control device of the infusion station is transferred to the new infusion device for the purpose of synchronization. In this case a conflict workflow may be initiated, in the course of which one or multiple parameters of the infusion operation may be adjusted in order to overcome a conflict with the reference patient data. For example, a user such as a physician or a nurse may be asked e.g. to confirm an adaption of the flow rate according to the newly received reference patient data, or to adapt a dose rate while continuing with the same flow rate.

The object is also achieved by means of a method for administering a medical fluid to a patient, the method comprising: providing an infusion station comprising a rack and a control device, the rack defining a multiplicity of slots, wherein the control device is configured to synchronize reference patient data between infusion devices attached to said multiplicity of slots, said reference patient data relating to a patient to be administered with a medical fluid using the infusion station; and attaching a new infusion device to one of said multiplicity of slots of the rack. Herein, the control device, on the occasion of attaching the new infusion device to one of the multiplicity of slots of the rack, receives, from the new infusion device, device patient data defined on the new infusion device, and updates said reference patient data based on the device patient data dependent on an infusion status of the new infusion device and on an infusion status of the infusion station.

The advantages and advantageous embodiments described above for the system equally apply also to the method, such that it shall be referred to the above.

The idea underlying the invention shall subsequently be described in more detail with reference to the embodiments shown in the figures. Herein:

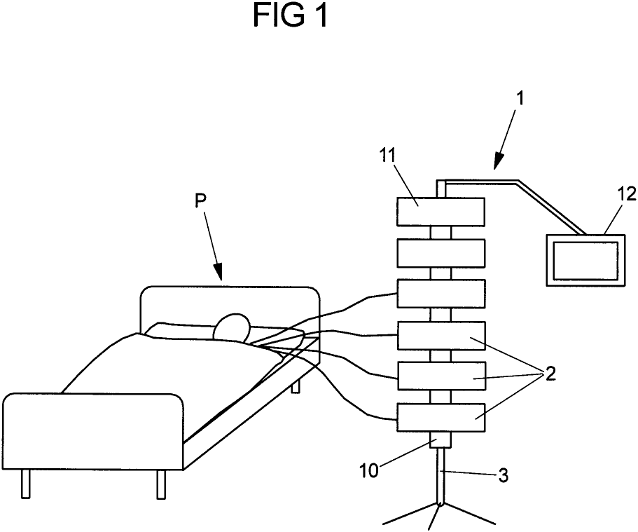

FIG. 1 shows a schematic drawing of an infusion station at the bedside of a patient;

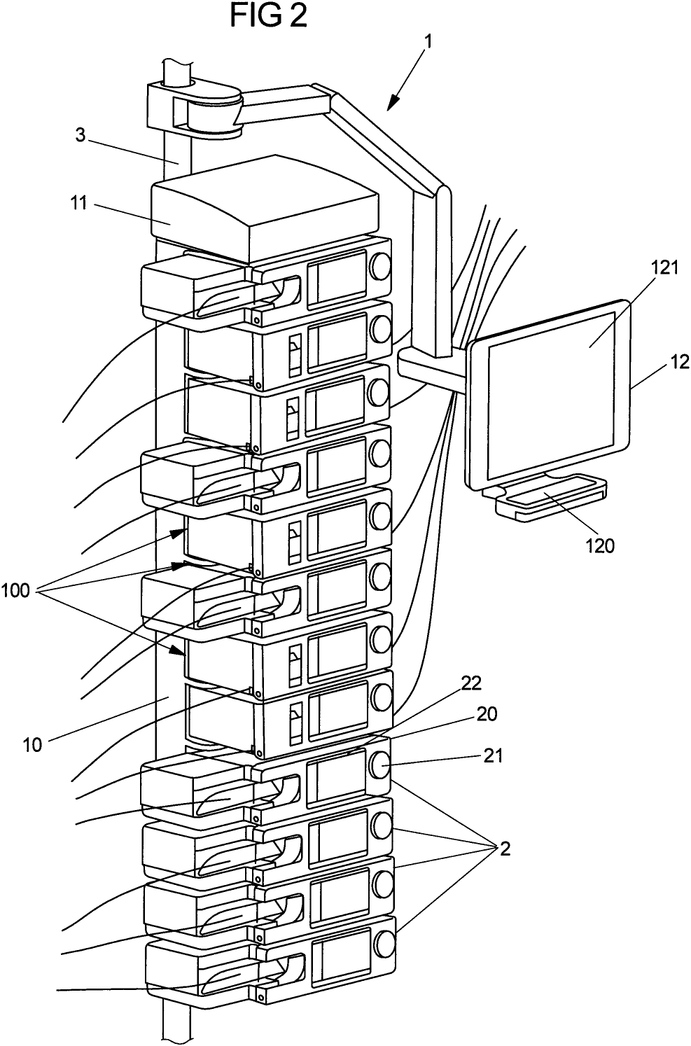

FIG. 2 shows a view of an embodiment of an infusion station;

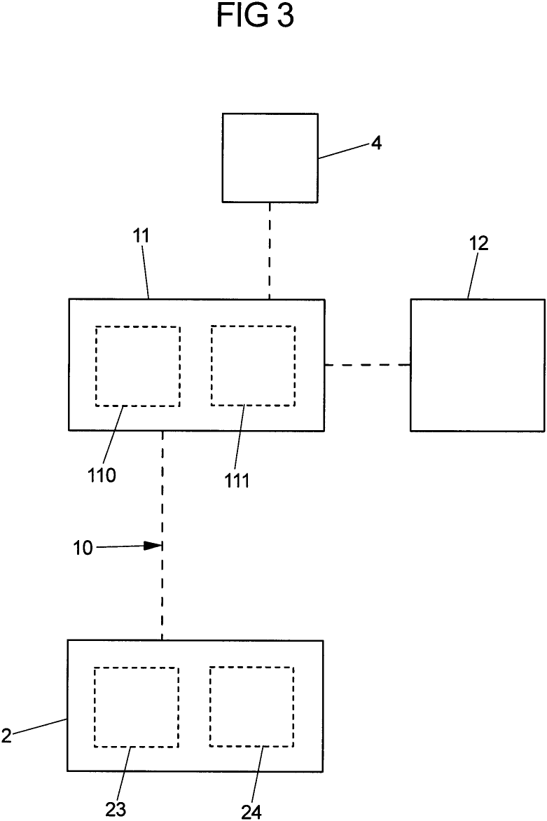

FIG. 3 shows a schematic diagram of the interaction of a control device and an infusion device of an infusion station;

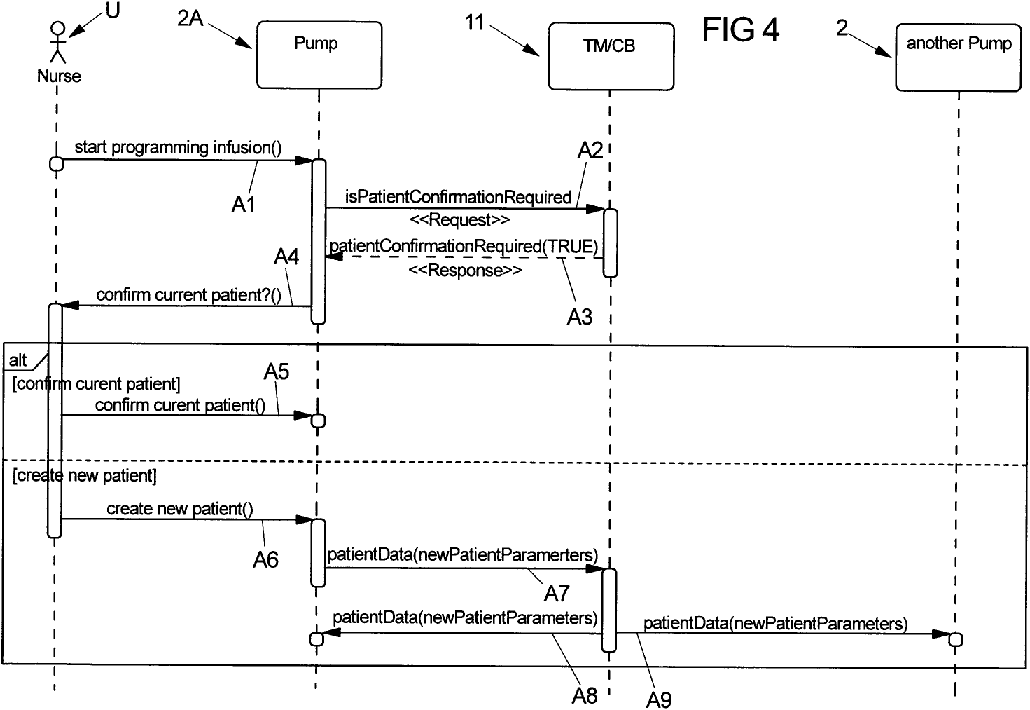

FIG. 4 shows an interaction chart for the communication in between an infusion device and a control device of an infusion station during the programming of an infusion operation by a user, in a situation in which the infusion station is not infusing;

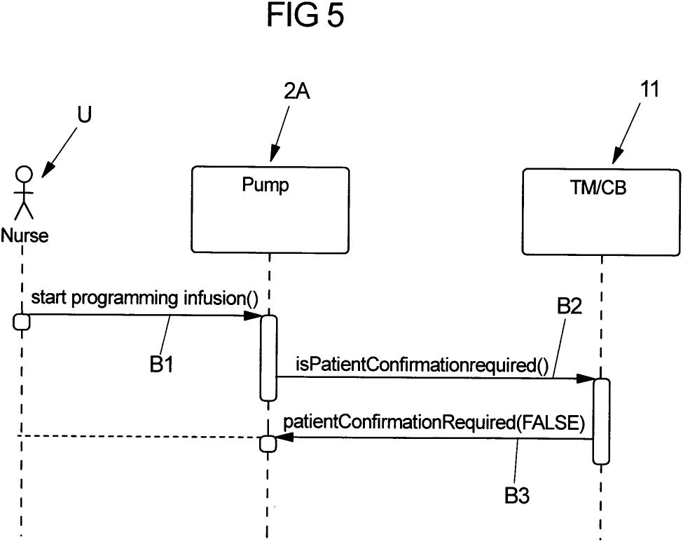

FIG. 5 shows an interaction chart for the communication between an infusion device and a control device of an infusion station during the programming of an infusion operation by a user, in a situation in which the infusion station is already infusing; and

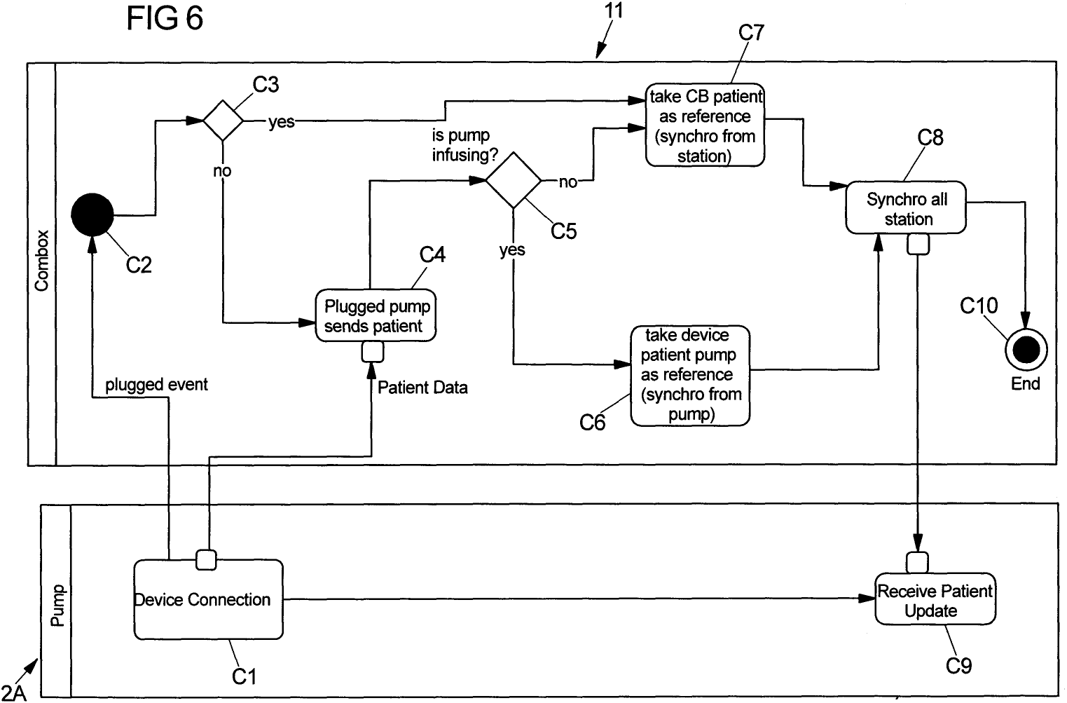

FIG. 6 shows a flow diagram for the synchronization of patient data in between an infusion device and a control device of an infusion station when attaching the infusion device to the infusion station.

FIG. 1 shows a schematic drawing of an infusion station 1 at the bedside of a patient P, for example in a healthcare environment, such as in an intensive care unit (ICU) of a hospital.

As visible from FIG. 1 in view of FIG. 2, the infusion station 1 comprises a rack 10 defining a multiplicity of slots 100 to which a multiplicity of infusion devices 2 may be attached. The rack 10, at the slots 100, provides for a mechanical and electrical connection for the infusion devices 2, the rack 10 serving as a communication link as well as a power supply such that the infusion devices 2 connected to the rack 10 are interconnected with each other and further are connected to a power supply via the rack 10.

The rack 10 comprises a control device 11 which, as schematically shown in FIG. 3, comprises a memory device 110 and a processing device 111 and hence comprises electronic circuitry for controlling operation of the infusion station 1. The control device 11 in particular provides for a connection to a hospital information system 4, such that via the control device 11 data may be transferred to and may be received from the hospital information system 4.

In particular, the control device 11 may be configured to send infusion data resulting from infusion operations carried out by infusion devices 2 of the infusion station 1 to the hospital information system 4. Alternatively or in addition, the control device 11 may be configured to receive configuration data, such as drug library data, from the hospital information system 4.

In addition, a nurse call system and a reading device such as a barcode reader or the like may be connected to the control device 11.

Alternatively or in addition, the control device 11 may be configured to control operation of the infusion devices 2 connected to the rack 10, for example for carrying out infusion operations in a concerted fashion, such as within a medical protocol for a multichannel infusion.

The rack 10 together with the control device 11 and the infusion devices 2 arranged thereon is connected to a stand 3 placed at the bedside of a patient P, as visible from FIG. 1. The rack 10 holds the infusion devices 2 connected thereto in an organized fashion, forming a vertical stack of infusion devices 2.

The infusion station 1 in addition comprises an interface device 12, also denoted as therapy manager, which is functionally connected to the control device 11 - as indicated in FIG. 3 - and comprises an input device 120 and a display 121. The interface device 12 is configured to output information about infusion operations carried out by infusion devices 2 of the infusion station 1. In addition, a user may enter information into the input device 120 for example for programming infusion operations of the infusion station 1.

As visible from FIG. 2, the infusion devices 2 may be configured as syringe infusion pumps or as volumetric (peristaltic) infusion pumps. In either case, the infusion devices 2 may comprise a housing 20, an input device 21 for example in the shape of a rotary knob and/or a keypad or the like for inputting data into the respective infusion device 2, for example for programming an infusion operation, and a display 22 for outputting information, for example relating to an ongoing infusion operation.

Referring again to FIG. 3, each infusion device 2 may comprise a memory device 23 for storing information and a processing device 24 for controlling operation of the infusion device 2.

The infusion devices 2 arranged on the rack 10 of the infusion station 1 may be configured to individually or together in a multichannel operation perform infusions on a patient P to which the infusion station 1 is assigned. Herein, each infusion station 1 generally is assigned to a single patient P, the infusion devices 2 hence being dedicated to the patient P for carrying out infusion operations on the patient P.

When setting up an infusion operation on an infusion device 2, generally patient data, such as the patient’s age, weight, gender, body surface area, body mass index and the like, is taken into account for defining certain settings of the infusion operation. For example, within a drug library a variety of different drugs may be associated with a variety of different limits, for example relating to a flow rate, a dosage, a dose rate or the like, wherein the limits may depend on patient data, for example the patient’s age or the patient’s weight. In addition, whereas a flow rate generally is defined as a fluid volume per time administered to a patient (stated for example in ml/h), a dose rate (also denoted as infusion rate) takes into account pharmacokinetics and refers to a rate at which a drug should be administered to achieve a steady-state of a particular dose within e.g. a patient’s plasma compartment, the dose rate herein generally being computed by taking patient data, in particular the patient’s weight, into account.

As the infusion station 1 with the infusion devices 2 connected to the rack 10 is assigned to a single patient, all infusion devices 2 of the infusion station 1 should use the same patient data. It hence is a task of the control device 11 to ensure that patient data, i.e. all data relating to the patient assigned to the infusion station 1, is synchronized in between the different infusion devices 2 attached to the rack 10 and the control device 11.

Referring now to FIG. 4, in case a user U, such as a physician or a nurse, wishes to program an infusion operation on a particular infusion device 2A (step A1), the infusion device 2A sends a message to the control device 11 to inquire from the control device 11 whether a patient confirmation is required (step A2). The control device 11 responds to the request of the infusion device 2A by sending a response message indicating to the infusion device 2A whether a patient confirmation is required or not, which in the instant case is true (step A3). The infusion device 2A hence indicates to the user U to either confirm the patient data currently defined on the infusion station 1 or to define a new patient by entering new patient data (step A4).

If the user U confirms the patient data as previously defined on the infusion station 1, the programming of the infusion operation takes place based on previously defined reference patient data as stored in the control device 11 (step A5).

In the alternative, if the user U decides to create a new patient (step A6), the user U enters new patient data into the infusion device 2A, which is forwarded by the infusion device 2A to the control device 11 (step A7). In the control device 11, previously stored reference patient data is replaced by the patient data received from the infusion device 2A, upon which the control device 11 distributes the new reference patient data to all infusion devices 2, 2A connected to the rack 10 and hence being part of the infusion station 1 (steps A8, A9). Hence, the newly defined patient data, entered by the user U in step A6, is transferred to the control device 11, which distributes the patient data as new reference patient data to all infusion devices 2, 2A connected to the rack 10.

An entering of new patient data on an infusion device 2A on which an infusion operation shall be programmed by a user U is possible, in one embodiment, if and only if the infusion station 1 is currently not infusing, i.e., none of the infusion devices 2 connected to the rack 10 other than the currently programmed infusion device 2A is currently infusing, that is if no infusion operation is currently carried out on the infusion station 1.

If, in contrast, an infusion operation is carried out by one or multiple of the infusion devices 2 of the infusion station 1, no entering of a new patient and no replacement of the current reference patient data is admissible. Referring now to FIG. 5, if a user U in this case initiates a programming of an infusion device 2A (step B1), the infusion device 2A sends a message to the control device 11 to inquire whether a patient confirmation is required (step B2). In this case the control device 11 responds by a message indicating that no patient confirmation is required, the response message hence having the value FALSE (step B3). The user U hence is not allowed to enter a new patient, but may continue with programming an infusion operation on the infusion device 2A based on the previously defined reference patient data.

Referring now to FIG. 6, upon attaching an infusion device 2A to a slot 100 of the rack 10 of the infusion station 1, a synchronization of patient data in between the infusion station 1 and the newly attached infusion device 2A must take place, as patient data defined on the infusion device 2A may differ from reference patient data defined in the control device 11 and previously attached infusion devices 2 of the infusion station 1. The synchronization herein takes place substantially immediately following the attachment of the infusion device 2A to the infusion station 1, and shall involve a minimum number of user interactions in order to obtain an easy, fast and comfortable synchronization procedure.

FIG. 6 shows a flow chart for performing a synchronization procedure of patient data involving the newly attached infusion device 2A and the control device 11 of the infusion station 1.

If an infusion device 2A is newly attached to a slot 100 of the rack 10 of the infusion station 1 (step C1 in FIG. 6), a connection event is identified at the control device 11 (step C2) such that the control device 11 becomes aware of the infusion device 2A having been connected to the rack 10. Following the connection event in step C2, the control device 11 checks whether the infusion station 1 is currently infusing (step C3). For this, the control device 11 examines whether one or multiple of the infusion devices 2 which previously had been connected to the rack 10 are currently carrying out an infusion operation.

If the infusion status of the infusion station 1 indicates that the infusion station 1 is currently not infusing, the control device 11 in step C4 receives device patient data from the infusion device 2A. In step C5 it is checked whether the infusion device 2A is currently carrying out an infusion operation. If this is the case, it is assumed that the device patient data should have priority over the previously stored reference patient data as defined in the control device 11, such that the device patient data replaces the reference patient data as previously stored in the control device 11 and hence becomes the new reference patient data (step C6).

If it in step C3 is found that the infusion station 1 is in an infusing state, i.e., one or multiple of the infusion devices 2 already previously connected to the rack 10 are carrying out an infusion operation, it is assumed that the reference patient data as previously stored in the control device 11 should have priority over device patient data which possibly is stored on the newly attached infusion device 2A, such that the reference patient data as previously stored in the control device 11 is maintained (step C7).

In step C8, the now present reference patient data, which may correspond to the updated reference patient data of step C6 or the previous reference patient data of step C7, is synchronized to all infusion devices 2, 2A connected to the rack 10 of the infusion station 1, such that all infusion devices 2, 2A receive the now valid reference patient data (step C9). This includes also the newly attached infusion device 2A, such that it is made sure that in any case all infusion devices 2, 2A are synchronized with the same reference patient data.

The process thereupon ends in step C10.

In case of attaching a new infusion device 2A to the infusion station 1, hence a synchronization procedure takes place which synchronizes the patient data between all infusion devices 2, 2A connected to the infusion station 1. Herein, dependent on an infusion status of the new infusion device 2A and an infusion status of the infusion station 1 including all previously connected infusion devices 2 are taken into account to determine whether the device patient data as stored on the new infusion device 2A or the reference patient data as previously defined in the control device 11 should be given priority. In any case, at the end of the synchronization procedure all infusion devices 2, 2A are set to the same reference patient data and hence make use of the same reference patient data for example for defining an infusion operation.

In the flow diagram of FIG. 6 the reference patient data as previously defined in the control device 11 is always given priority if the infusion station 1 is in an infusing state, i.e. one or multiple of the infusion devices 2 previously connected to the infusion station 1 are currently infusing. If the infusion station 1 is in an infusing state, the reference patient data as previously defined is maintained, independent on whether the new infusion device 2A is infusing or not.

If, in this case, the new infusion device 2A is infusing, reference patient data as previously defined in the control device 11 is transferred to the new infusion device 2A, causing an updating of the patient data as defined in the infusion device 2A. This may give rise to a conflict with the ongoing infusion operation on the infusion device 2A, upon which a conflict workflow may be carried out on the infusion device 2A. During the conflict workflow, a user U may be asked for a confirmation for an adaption of parameters of the infusion operation, for example for a confirmation of an adapted flow rate or an adapted dose rate.

The idea underlying the invention is not limited to the embodiments described above, but may be implemented in a different fashion.

Reference patient data may relate to a specific patient currently treated by an infusion station. Reference patient data however may also refer to a generic, default patient, assuming some average, standard values for the patient, for example the patient’s weight.

It is conceivable that multiple racks are present within an infusion station and are associated with a single control device for controlling the racks and the infusion devices arranged thereon. Synchronization of patient data herein may be effected by a single control device associated with the multiple racks for all infusion devices arranged on the racks.

### LIST OF REFERENCE NUMERALS

1 Infusion station
10 Rack
100 Slot
11 Control device
110 Memory device
111 Processing device
12 Interface device
120 Input device
121 Display
2, 2A Infusion device
20 Housing
21 Input device
22 Display
23 Memory device
24 Processing device
3 Stand
4 Hospital information system
A1-A9 Steps
B1-B3 Steps
C1-C10 Steps
P Patient
U User

1. A system for administering a medical fluid to a patient, comprising:
an infusion station comprising a rack and a control device, the rack defining a multiplicity of slots,
wherein the control device is configured to synchronize reference patient data between infusion devices attached to said multiplicity of slots, said reference patient data relating to a patient to be administered with a medical fluid using the infusion station, and
a new infusion device to be attached to one of said multiplicity of slots of the rack,
wherein the control device is configured, on an occasion of attaching the new infusion device to one of the multiplicity of slots of the rack, to receive, from the new infusion device, device patient data defined on the new infusion device, and to update said reference patient data based on the device patient data dependent on an infusion status of the new infusion device and on an infusion status of the infusion station.

2. The system according to claim 1, wherein the control device is configured to identify a connection event on the occasion of attaching the new infusion device to one of the multiplicity of slots of the rack, and to update said reference patient data based on the device patient data subsequent to the connection event.

3. The system according to claim 1, wherein the new infusion device is configured to transfer said device patient data to the control device on the occasion of attaching the new infusion device to one of the multiplicity of slots of the rack.

4. The system according to claim 1, wherein the control device is configured to synchronize the updated reference patient data between the infusion devices attached to said multiplicity of slots.

5. The system according to claim 1, wherein the infusion status of the new infusion device is indicative of whether the new infusion device is currently carrying out an infusion operation.

6. The system according to claim 1, wherein the infusion status of the infusion station is indicative of whether at least one of the infusion devices attached to said multiplicity of slots is currently carrying out an infusion operation.

7. The system according to claim 1, wherein the control device is configured to set the reference patient data to the device patient data dependent on the infusion status of the new infusion device and on the infusion status of the infusion station.

8. The system according to claim 1, wherein the control device is configured to set the reference patient data to the device patient data if the infusion status of the new infusion device indicates that the new infusion device is currently carrying out an infusion operation, and if in addition the infusion status of the infusion station indicates that none of the infusion devices attached to said multiplicity of slots is currently carrying out an infusion operation.

9. The system according to claim 1, wherein the control device is configured to not update said reference patient data if the infusion status of the new infusion device indicates that the new infusion device is currently not carrying out an infusion operation, or if the infusion status of the infusion station indicates that at least one of the infusion devices attached to said multiplicity of slots is currently carrying out an infusion operation.

10. The system according to claim 1, wherein the control device is configured to transfer said reference patient data to the new infusion device if the infusion status of the new infusion device indicates that the new infusion device is currently not carrying out an infusion operation, or if the infusion status of the infusion station indicates that at least one of the infusion devices attached to said multiplicity of slots is currently carrying out an infusion operation.

11. The system according to claim 1, wherein the new infusion device is configured, in case the new infusion device is currently carrying out an infusion operation, to initiate a conflict workflow for adjusting at least one parameter of the infusion operation according to the reference patient data.

12. The system according to claim 11, wherein the new infusion device is configured, within the conflict workflow, to adjust at least one parameter of the infusion operation upon a user confirmation.

13. A method for administering a medical fluid to a patient, comprising:
providing an infusion station comprising a rack and a control device, the rack defining a multiplicity of slots, wherein the control device is configured to synchronize reference patient data between infusion devices attached to said multiplicity of slots, said reference patient data relating to a patient to be administered with a medical fluid using the infusion station, and
attaching a new infusion device to one of said multiplicity of slots of the rack,
wherein the control device, on an occasion of attaching the new infusion device to one of the multiplicity of slots of the rack , receives, from the new infusion device, device patient data defined on the new infusion device, and updates said reference patient data based on the device patient data dependent on an infusion status of the new infusion device and on an infusion status of the infusion station.
